(19) United States
(12) Patent Application Publication
(10) Pub. No.: US 2020/0277556 A1
(43) Pub. Date: Sep. 3, 2020

(54) USE OF AN ADDITIVE TO IMPROVE THE QUALITY OF BEVERAGES BASED ON PLANT MUSTS

(71) Applicants: OENOTROPIC INNOVATION, Saint Gilles Les Bains (FR); INSTITUT NATIONAL DE LA RECHERCHE AGRONOMIQUE, Paris (FR); INSTITUT NATIONAL D'ETUDES SUPERIEURES AGRONOMIQUES DE MONTPELLIER, Montpellier (FR)

(72) Inventors: François-Xavier SAUVAGE, Les Matelles (FR); Sacha BAUDOIN, Saint Gilles Les Bains (FR); Aude VERNET, Montpellier (FR); Amélie BREYSSE, Montpellier (FR); Julie RETTMAN, Villenave D'ornon (FR)

(73) Assignees: OENOTROPIC INNOVATION, Saint Gilles Les Bains (FR); INSTITUT NATIONAL DE LA RECHERCHE AGRONOMIQUE, Paris (FR); INSTITUT NATIONAL D'ETUDES SUPERIEURES AGRONOMIQUES DE MONTPELLIER, Montpellier (FR)

(21) Appl. No.: 16/649,297

(22) PCT Filed: Sep. 26, 2018
(86) PCT No.: PCT/FR2018/052366

§ 371 Date: Mar. 20, 2020

(30) Foreign Application Priority Data
Sep. 26, 2017 (FR) 17/58872

(51) Int. Cl. C12H 1/00; C12N 11/00; C12P 21/06; A23J 3/34; A23L 2/62; A23L 2/84

(52) U.S. Cl. CPC C12H 1/003; C12N 11/00; C12P 21/06; A23V 2002/00; A23L 2/62; A23L 2/84; A23J 3/346

(57) ABSTRACT

The present disclosure relates to an additive for treating beverages made from plant musts, particularly wine, beer, cider or fruit juices, and more particularly for improving the quality of such beverages, namely, for example, protein stability and for avoiding the cloudiness of such beverages. In its broadest application, such an additive includes a cysteine protease and a cofactor.

### CROSS-REFERENCE TO RELATED APPLICATIONS

This application is a National Phase entry of International Patent Application No. PCT/FR2018/052366, filed Sep. 26, 2018, which claims priority to French Patent Application No. 17/58872, filed Sep. 26, 2017, both of which are incorporated by reference herein.

### TECHNICAL FIELD

The object of the present invention is an additive for treating beverages based on plant musts, in particular wine, beer, cider or fruit juices, and more particularly for improving the quality of such beverages, namely in particular protein stability, and for avoiding the cloudiness of such beverages, generally due to the presence of yeasts, of particles in suspension, mainly proteins, which may have very different sizes and compositions.

### BACKGROUND

It is well known that the marketing of the above-mentioned beverages in bottles not only requires their being clear at the time of bottling, but that they remain so over time, especially for wines that are kept for relatively long periods of time. However, it is known that current wine stabilisation treatments are unsatisfactory, due to precipitation of tartaric or protein salts, known in the oenology profession as “tartaric and protein breakage”. A known solution consists in treating musts and wines with bentonite, but the doses required according to the needs can be sufficiently high to impoverish the organoleptic characteristics of the wines thus treated. Enzymatic tests were conducted using exogenous or yeast proteases to remove the proteins responsible for protein breakdown, but the results were not satisfactory.

The article “Effect of free and immobilised stem bromelain on protein hazein white wine”, authors I. BENUCCI, M. ESTI and K. LIBURDIN published in the Australian Journal of Grape and Wine Research 20, 347-352, 2014 is known in the state of the art.

Also known is the U.S. Patent Publication No. 2003/0165592 describing a winemaking process using a protease to remove heat-unstable proteins that cause a heat-induced precipitate; this document states that the introduction of a protease at the beginning of fermentation, prior to the generation of inhibitory factors, avoids the use of bentonite normally required for wine stabilisation. This document also states that protease can be used as an anti-foaming agent in fruit juices and during the fermentation of fruit juices. It cites different sources of proteases: microbial, plant and/or animal, the protease having to maintain sufficient activity at the pH of the fruit (between 2.5 and 4.0 approximately) to eliminate heat-unstable proteins, in particular:
fungal proteases from sources such as Aspergillus niger, Aspergillus oryzae, Rhizomucor meihei and Neosartorya fischeri;
yeast proteases from sources such as Candida olea and Saccharomyces cerevisiae; Bacterial proteases from Bacillus subtilis, or Bacillus lichenifomis;
animal proteinases pepsin and trypsin from cattle or pigs.

Among all these solutions, plant proteases are preferred in food beverages. However, it is recognized in the prior art, and in particular in U.S. Patent Publication No. 2003/0165592, that the following plant proteases: Ficus spp. ficin, Carica papaya papain, Ananas comosus or Ananas bracteratus bromelain, do not show good activity at an acid pH of the fruit and are therefore not expected to be able to hydrolyze fruit proteins. It is stated that bromelain and/or papain (obtained from Valley Research, Inc., South Bend, Ind.) were used to compare with the activity of proteases produced by A. Niger. It therefore appears from the prior art that bromelain does not give satisfactory results for the stabilisation of beverages based on plant musts, and more generally for improving the quality thereof.

### SUMMARY

Surprisingly, the inventors observed that the combination of a cysteine protease, such as bromelain, and a cofactor such as a sulphur agent or a calcium salt, makes it possible to overcome the drawbacks of the prior art and to offer a particularly active protein-stabilizing additive. Thus, in its broadest application, the invention relates to an additive for improving the quality of beverages based on plant musts comprising a cysteine protease and a cofactor.

A first object of the invention relates to the use of an additive comprising a cysteine protease and a cofactor to improve the quality of beverages based on plant musts by increasing protein hydrolysis. Cysteine protease can be of any type: of plant, animal, bacterial or synthetic origin. Cysteine proteases of plant origin include proteases extracted from various fruit such as pineapples, papayas, figs, kiwis, ginger . . . . More generally, cysteine proteases include cathepsins, caspases, calpains.

In a preferred embodiment, the cysteine protease is plant-derived. Such a protease can be bromelain extracted from pineapple, either extracted from a pineapple fruit (excluding leaves, stems and roots) or extracted from the stem (leaves, stems and roots). In a very preferred embodiment, bromelain is extracted from a fruit. Thus, in a particular embodiment, the invention relates to the use of an additive for the protein stabilisation of beverages based on plant musts comprising bromelain, characterised in that bromelain is extracted from pineapple fruit and in that it additionally contains a sulphur-containing amino acid or a peptide comprising a sulphur-containing amino acid.

“Extracted from a pineapple fruit” within the meaning of this invention means that said variety of bromelain is obtained by extraction from a pineapple fruit alone, comprising the skin and the pulp, after removal of the leaves, stems and roots. This method of extraction is not usual, the tradition being rather to use pineapple waste for the extraction of bromelain, or specifically pineapple stems. As a matter of fact, in traditional medicine, in Hawaii, Japan and Taiwan, the latex (sap) from pineapple stems is used to clean wounds and burns and accelerate their healing, as well as to aid digestion and to treat certain cancers. In 1957, Ralph Heinicke of the Pineapple Research Institute discovered that the stems of pineapple plants, until then considered mere production residues, were rich in this enzyme. Europeans became interested in bromelain as early as the 1960s. Today, it is used on this continent to speed healing after surgery or sports injuries as well as to treat phlebitis and sinusitis and for more than 30 years, bromelain has also been used by the food industry to tenderize meat. For these reasons, the bromelain marketed is generally derived from pineapple waste, mainly the stems.

In another preferred embodiment, cysteine protease is extracted from ginger. The cofactor used to potentiate the activity of the cysteine protease may be a sulphur compound or a calcium salt.

In a preferred embodiment, said sulfur-containing amino acid or peptide is cysteine, methionine, glutathione, N-acetylcysteine, y-Glu-Cys (y-glutamylcysteine) or Cys-Glu (cysteinylglutamic acid). Another preferred embodiment, calcium salt is calcium chloride. The beverage based on plant musts can be beer, wine (white, rosé, red), fruit juice, cider, etc. . . . .

“Improvement of the quality of beverages based on plant musts” means improvements associated with protein hydrolysis such as improved protein stability (or reduced protein instability), and/or in the case of alcoholic beverages, improved alcoholic fermentation. The determination of the quantity of cofactors present in the beverage based on plant musts may be carried out either at the time of the addition of the additive or beforehand by type of beverage. In the latter case, it is possible to prepare in advance additives adapted to the different types of beverages based on plant musts and to add them without any step in the dosing of the quantity of cofactors.

The cofactor can be added to the reaction medium according to three (non-limiting) protocols:

(i) by addition during the grinding/extraction step as defined in Example 2, step 1; or

(ii) by addition during or after the concentration/filtration step as defined in Example 2, step 2, 3 or 4; or

(iii) by addition to the reaction medium, before, after or simultaneously with the addition of pineapple extract.

When the cofactor is a sulphur-containing amino acid or peptide, it can be added in the form of powder or extracts rich in sulphur-containing amino acid (dry yeast barks, cruciferers, garlic, onion, cabbage, etc.). When the cofactor is a sulphur-containing amino acid or peptide, its presence in the reaction medium is obtainable by conversion or bioconversion in the reaction medium of precursors of said sulphur-containing amino acid or peptide.

When the additive and the beverage to be treated are brought into contact, it is possible to:
add the protease and the cofactor in a free form directly into the beverage;
immobilize the protease or the cofactor on a support such as resin, bentonite, agar-agar . . . .

As regards the conditions for implementing the treatment of plant musts in accordance with the invention, the inventors have demonstrated a significant influence of the pH of the plant must on the activity of the additive. In particular, the addition of the additive may change the pH of the must. Generally speaking, the lower the pH of the wine, the more the dose of cofactor will have to be increased to optimise protein hydrolysis.

The skilled craftsman will know how to adapt the dose of cofactor according to the pH of the reaction medium and the must to be treated. As an indication, for an efficient use of the additive in the wine, the pH is advantageously but non-limitatively between 3 and 4, after addition of the additive. The pH of the medium should preferably be between 3.2 and 3.8, and even more preferably close to or equal to 3.8 after the additive has been added to the wine. For the use of the additive in beer, the pH of the medium should preferably be between 4 and 6 after addition of the additive.

A second object of the invention relates to an additive for improving the quality of beverages based on plant musts, comprising a cysteine protease and a sulphur compound other than cysteine. In one preferred embodiment, the sulphur compound is selected from glutathione and methionine.

As examples of particular additives according to the invention, we can cite the following associations:
Bromelain and glutathione;
Bromelain and methionine;
Bromelain and sulfur compound other than cysteine;
Cysteine protease extracted from ginger and glutathione;
Cysteine protease extracted from ginger and methionine;
Cysteine protease and sulfur compound other than cysteine.

A third object of the invention relates to a method for preparing an additive for improving the quality of beverages based on plant musts. In a special embodiment in which the cysteine protease is extracted from pineapple fruit, this method comprises the following steps:
grinding fresh pineapple fruit with a buffer of pH above 5, preferably pH 7, and a peptide or sulphur-containing amino acid (fruit may, for example, be spoiled fruit unfit for marketing);
centrifuging at a temperature ranging from 2° C. to 8° C.;
clarification and ultrafiltration.
   These pH and temperature ranges are only given as an indication and not as a limitation, and are only one embodiment considered as advantageous.

The buffer strength is advantageously between 0.05 and 1 Mol, preferably 0.1 Mol. The man of the trade will be able to determine the optimal force by routine experiments, by successive tests with various forces. In a preferred embodiment where the additive comprises bromelain extracted from pineapple fruit and cysteine, the preparation of the additive is carried out in 0.1M phosphate buffer with cysteine at 15 mM and pH 7. The additive thus prepared is useful in particular for the protein stabilisation of beverages based on plant musts or for improving the alcoholic fermentation of alcoholic beverages based on plant musts.

A fourth object of the invention relates to a method for improving the quality of beverages based on plant musts by increasing protein hydrolysis, consisting in adding a dose compatible with the doses usual in oenology and brewing of an additive comprising a cysteine protease and a cofactor, for one litre of fruit juice, beer or wine. This dose may range from 1 mg/L to 10000 mg/L. Advantageously but not restrictively, this dose is between 0.5 g/HI and 300 g/HI, i.e. between 5 mg/l and 3000 mg/l. In another embodiment of interest, it is between 1 mg/l and 100 mg/l.

In a particular embodiment in which a “crude” or “pre-purified” extract as defined in example 2 of a cysteine protease is used, the method for improving the quality of beverages based on plant musts by increasing protein hydrolysis consists in adding a dose between 1 mg/ml and 30 mg/ml, preferably between 5 mg/ml and 30 mg/ml, of an additive comprising a cysteine protease and a cofactor, for one litre of fruit juice, beer or wine. In another preferred embodiment, the method according to the invention is a method for protein stabilisation of beverages based on plant musts consisting in adding a dose between 1 mg/ml and 30 mg/ml, preferably between 5 mg/ml and 30 mg/ml, of a “crude” or “pre-purified” additive comprising bromelain, for one litre of fruit juice, beer or wine, characterized in that bromelain is extracted from pineapple fruit and in that it additionally contains a sulphur-containing amino acid or a peptide comprising a sulphur-containing amino acid. It should be noted that the sulphur compound can be provided by a pineapple extract which contains it naturally and/or by addition from an external source.

In general, the more bromelain extract is purified, the less sulphur compound, especially cysteine, it contains and the more it will need to be added to prepare the additive. These parameters can easily be adjusted by the person in the trade. The following are examples that illustrate, but are not limited to, the invention.

### BRIEF DESCRIPTION OF THE DRAWINGS

FIG. 1 shows the electrophoretic profile of a sample of white wine treated with different concentrations of additive according to the invention (VB=white wine; EFT=tropical fruit extract);

FIGS. 2 and 3 show the variation in grape protein of the control according to the treatments applied; and

FIGS. 4, 5 and 6 show the decrease in the amount of wine protein as a function of pH in the presence of an additive according to the invention, FIG. 4 at pH 3, FIG. 5 at pH 3.4 and FIG. 6 at pH 3.8.

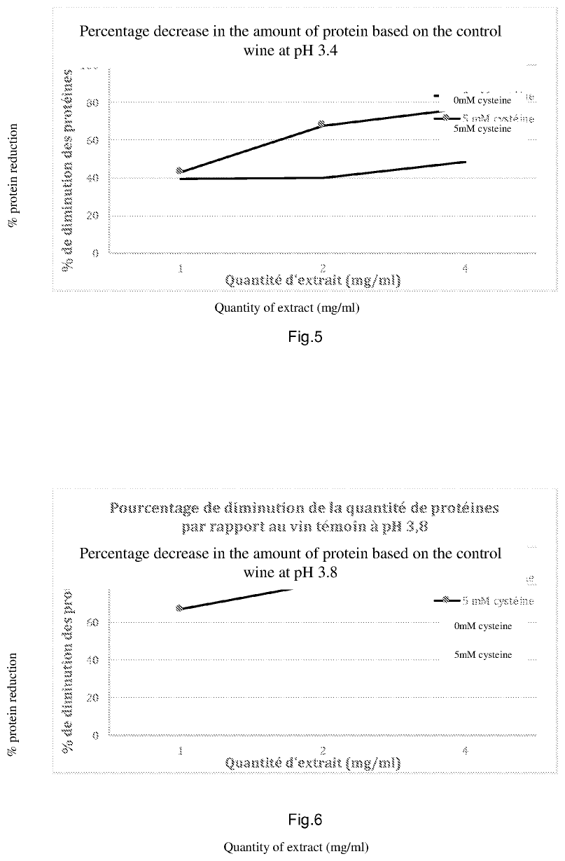

### DETAILED DESCRIPTION

### Example 1: Selection of an Optimal Form of Bromelain

One of the aspects of the present invention relates to the choice of an optimal form of bromelain as a cysteine protease used in the additive for beverage based on plant musts according to the invention. Table 1 below presents the different forms of bromelain (cysteine proteinases) from the Ananas comosus species listed by the inventors:

| | | | | | |
|---|---|---|---|---|---|
| | | Molecular | Iso- | | |
| Scientific or | Abbre- | mass in | electric | | Glyco- |
| trade name | viation | Dalton | point | Sequence | sylation |

Bromelain extracted from the stem

| | | | | | |
|---|---|---|---|---|---|
| Stem | F4 and | 2800 | >10 | 212 amino | Glyco- |
| Bromelain | F5 | (sequence + | | acids | sylated |
| EC 3.4.22.32 | | sugar) | | | |
| Ananain | F9 | 23464 | >10 | 216 amino | Not |
| EC 3.4.22.31 | | (sequence) | | acids | Glyco- |
| | | | | | sylated |
| Comosain | F9/b | 24509 and | >10 | N-term | Glyco- |
| | | 23569 | | | sylated |
| | SBA/a | 23550 and | 4.8 and | N-term | Glyco- |
| | and | 3560 | 4.9 | | sylated |
| | SBA/b | | | | |

Bromelain extracted from the fruit

| | | | | |
|---|---|---|---|---|
| Fruit | 23000 | 4.6 | N-term | Non- |
| bromelain | | | | glyco- |
| EC 3.4.22.33 | | | | sylated |

The bromelain which is the subject of this invention may be extracted from pineapple fruit. However, the latter contains two types of bromelain: mainly the “fruit bromelain” type but also the “stem bromelain” type, which is much less present.

### Example 2: Extraction and Pre-Purification by Filtration and Ultrafiltration of Enzymatic Activity

The following description gives a non-limiting example of a method for preparing a pineapple extract containing bromelain and cysteine which can be used as an additive according to the invention.

Step 1: grinding and extraction
Prepare 100 g of fresh pineapple waste plus 200 ml of 0.1M phosphate buffer at pH 7 to which 15 mM cysteine is added.
Grind (stainless steel fin mill) at maximum speed for 1 minute.
Leave to rest for 1 hour.
Centrifuge at 8000 rpm for 20 minutes at +4° C.

Step 2: freeze-drying of the above-mentioned dry extract.

Step 3: Ultrafiltration
Recover the supernatant after centrifugation.
Make a dialysis against a 0.01M phosphate buffer at pH 7 using one membrane with a cut-off threshold of 10 kDa.
Concentrate five times.

Step 4: freeze-drying the dry extract

The succession of steps 1 and 2 leads to an extract called “crude D”. The succession of steps 1, 3 and 4 leads to an extract called “pre-purified D”.

### Example 3: Effect of Cysteine on the Activity of Bromelain in the Hydrolysis of Beer Wort Proteins

Protein hydrolysis tests using cysteine proteases have been carried out on beer. The case of beer is a little peculiar. As a matter of fact, the use of bromelain to hydrolyse proteins in beer is known from the prior art.

Unexpectedly, however, the inventors demonstrated that the addition of cysteine to a beer wort still improves protein hydrolysis compared to adding bromelain alone. This result obtained by gel migration of protein preparations was performed using a “crude” bromelain extract as defined in example 2 and comparing the size of the proteins contained in the following 4 mixtures:

a. 1 ml untreated beer wort alone;

b. 1 ml beer wort+12.5 mg “crude” bromelain extract;

c. 1 ml beer wort+12.5 mg “crude” bromelain extract+1 mM cysteine;

d. 1 ml beer wort+12.5 mg “crude” bromelain extract+10 mM cysteine.

After migration on SDS PAGE gel, the protein bands from tests b, c and d were compared to those from test a. The revelation of the protein profile from gel test d. shows a greater hydrolysis of the proteins in the beer wort. Namely a more pronounced disappearance of the protein bands. These results validated the positive effect of cysteine as a cofactor on the activity of bromelain in beer wort (gel results are not shown).

### Example 4: Effect of Different Cysteine Proteases on the Hydrolysis of Beer Wort Proteins

Experiments were carried out to validate the possibility of extending the results obtained using bromelain to hydrolyse plant must proteins to other cysteine proteases. The cysteine protease tested is extracted from ginger. With the exception of step 1 where no cysteine was added during extraction, the ginger extract was obtained using the same protocol as that used to prepare the “crude” bromelain extract used in Example 3.

Experiments were conducted with ginger extracts under the following conditions:

a. 1 ml untreated beer wort alone;

b. 1 ml beer wort+12.5 mg “crude” ginger extract;

c. 1 ml beer wort+12.5 mg “crude” ginger extract+10 mM cysteine.

After migration on SDS PAGE gel, the protein bands from tests b and c were compared to test a. The revelation of the protein profile from gel test c. shows a greater hydrolysis of the proteins in the beer wort, i.e. a more pronounced disappearance of the protein bands. These results validate the capacity of the cysteine protease extracted from ginger to hydrolyze beer wort proteins in the presence of the cofactor, cysteine (gel results are not shown).

Conclusion: These results validate the fact that not only bromelain but also the cysteine protease extracted from ginger are capable of hydrolysing the proteins of plant musts. This result supports the hypothesis that all cysteine proteases, especially those derived from fruit, can be used with a cofactor to hydrolyze proteins from plant musts. These results pave the way for new uses of cysteine proteases with their cofactors for protein hydrolysis, particularly in the context of protein stabilization in beverages based on plant musts.

### Example 5: Effect of the Additive on a White Wine

The interest of using an additive according to the invention has also been validated on wine. By way of example, an additive according to the invention may comprise a main active compound consisting of:
bromelain extracted from pineapple fruit (not from the stems, leaves or roots);
a peptide or an amino acid having a sulphur compound, in particular cysteine, methionine or glutathione, or a calcium salt, in particular calcium chloride.

The addition of sulphur-containing amino acid can be done in the form of powder or the addition of extracts rich in sulphur-containing amino acid (dry yeast bark, cruciferer, garlic, onion, cabbage, . . . ).
1) Effect of a “crude” extract of bromelain on a Sauvignon type white wine with a pH of 3.25

The operation is as follows:

Preparation:
Wine: Sauvignon type white wine with a pH of 3.25.
Weigh 3 different quantities of crude extract as obtained by the above method
25.5 mg to be dissolved in 0.9 ml of white wine
12.5 mg to be dissolved in 0.9 ml of white wine.
5 mg to be dissolved in 0.9 ml of white wine.
25.5 mg to be dissolved in 0.9 ml of water.
Leave in contact for 16 hours, with the plate stirring at 80 rpm and at room temperature.

Adsorption and analysis of proteins after hydrolysis:
Then add 0.1 ml of 10 g/l bentonite (electra).
Wheel for 30 minutes at 40 rpm.
Centrifuge for 10 minutes at 7000 rpm at 10° C.
Place the sediment in 0.05 ml of Laemmli 1×.
Place on a stirring plate for 16 hours at room temperature.
Centrifuge for 10 minutes at 7000 rpm at 10° C.
Perform electrophoresis analysis with the Experion® device.

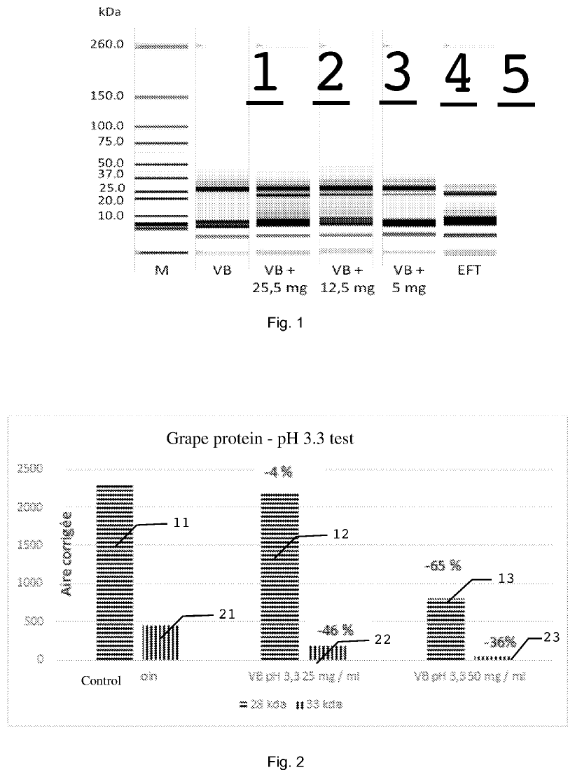

FIG. 1 shows the electrophoretic profiles of a sample of white wine, with the profile (1) of the wine without treatment, and the profiles (2 to 4) of the wine after treatment with an additive according to the invention, at concentrations of 25.5, 12.5, 5 mg/ml respectively. The profile (5) corresponds to the profile of the above extract, with a concentration of 5 mg/ml in water. The profiles were established by EXPERION (trade name) electrophoresis equipment.

The results show that the proteins in the wine have been partially hydrolysed. The samples treated with the additive according to the invention have the two bands associated with grape proteins (approximately 28 and 33 kDa) and also a band below 25 kDa which corresponds to the molecular weight of bromelain.
2) Effect of a “crude” extract of bromelain on a Sauvignon type white wine with a pH of 3.34 and a Sauvignon white wine with a pH adjusted to 3.8

Preparation:
Wine: Sauvignon type white wine with a pH of 3.34 and Sauvignon white wine with a pH adjusted to 3.8.
Weigh two different quantities of crude extract as obtained with the above method:
50 mg to be dissolved in 0.9 ml of white wine
25.5 mg to be dissolved in 0.9 ml of water.
Leave in contact for 16 hours, with the plate stirring at 80 rpm and at room temperature.

Adsorption and Analysis of Proteins after Hydrolysis:
Then add 0.1 ml of 10 g/l bentonite (electra).
Wheel for 30 minutes at 40 rpm.
Centrifuge for 10 minutes at 7000 rpm at 10° C.
Place the sediment in 0.05 ml of Laemmli 1×.
Place on a stirring plate for 16 hours at room temperature.
Centrifuge for 10 minutes at 7000 rpm at 10° C.
Perform electrophoresis analysis with the Experion® device.

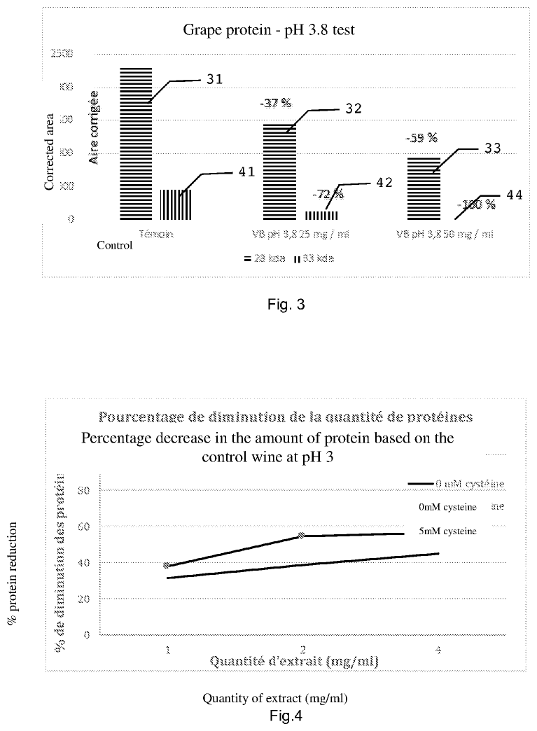

FIG. 2 shows the protein profiles with two molecular weights, 28 Kda and 33 Kda, for the first Sauvignon wine at pH 3.34. The peaks (11, 12 and 13) quantify the protein content by weight of 28 Kda respectively of the control without additive, the wine at pH 3.34 treated with 25.5 mg/ml of additive and the wine treated with 50 mg/ml of additive. The peaks (21, 22 and 23) quantify the protein content by weight of 33 Kda respectively of the control without additive, the wine at pH 3.34 treated with 25.5 mg/ml of additive and the wine treated with 50 mg/ml of additive. In all cases, a reduction in protein content is observed when the additive is added, with differentiated effects according to the molecular weight.

FIG. 3 shows the protein profiles with two molecular weights, 28 Kda and 33 Kda, for the first Sauvignon wine at pH 3.8. The peaks (41, 32 and 33) quantify the protein content by weight of 28 Kda respectively of the control without additive, the wine at pH 3.8 treated with 25.5 mg/ml of additive and the wine treated with 50 mg/ml of additive. The peaks (41, 42 and 43) quantify the protein content by weight of 33 Kda respectively of the control without additive, the wine at pH 3.8 treated with 25.5 mg/ml of additive and the wine treated with 50 mg/ml of additive.

In all cases a reduction in the protein content is observed when the additive is added, with differentiated effects according to molecular weight, more markedly than for wine at pH 3.34.
3) Effect of a “pre-purified” extract of bromelain on a Gewurztraminer type wine at pH 3.8/3.4 and 3

These results were produced in the framework of the experimental design reproduced in Table 2 below:

| Factors | Terms and conditions | | |
|---|---|---|---|
| amount of extract (mg/ml) | 1 | 2 | 4 |
| amount of cysteine (mM) | | 5 | 10 |
| wine pH | 3 | 3.4 | 3.8 |

Wine: Gewurztraminer type with a pH of 3.8 and with a pH adjusted to 3 and 3.4;
Weigh two different amounts of cysteine and three different amounts of pre-purified extract as obtained by the above method;
Dissolve in the white wine and leave in contact for 16 hours, with the plate stirring at 80 rpm and at room temperature.

Adsorption and Analysis of Proteins after Hydrolysis:
Then add 0.1 ml of 10 g/l bentonite (electra);
Wheel for 30 minutes at 40 rpm;
Centrifuge for 10 minutes at 7000 rpm at 10° C.;
Place the sediment in 0.05 ml of Laemmli 1×;
Place on a stirring plate for 16 hours at room temperature;
Centrifuge for 10 minutes at 7000 rpm at 10° C.;
Take 60 μl of the supernatant;
Add 20 μl of Laemmli 4×;
Place 50 μl on SDS PAGE gel.

The results are shown in Tables 4, 5 and 6. On the one hand, there is a faster and more efficient hydrolysis of proteins in the presence of cysteine, as described above. On the other hand, these data highlight the influence of pH on hydrolysis and show that for wine, the optimal pH is 3.8.

### Example 6: Evaluation of the Increase in Bromelain Activity as a Function of the Added Cofactor

Cysteine is known to improve the activity of bromelain. In this experiment, the inventors compared the effect of three other cofactors on the activity of bromelain: methionine, glutathione and CaCl₂). To test the relevance of using methionine and glutathione as cofactors of a cysteine protease, the inventors compared the effect of these cofactors to the effect of cysteine on the activity of bromelain in wine.

To this end, the following protocol has been implemented:
Wine: Gewurztraminer type with a pH of 3.8;
Add 10 mM of cysteine, glutathione or methionine, for 3 wine samples;
Weigh four times 8 mg of the pre-purified extract obtained from the sequence of steps 1, 2 and 4 of the above method;
Dissolve each pre-purified extract in wine, wine in the presence of cysteine, wine in the presence of glutathione and wine in the presence of methionine;
Leave in contact for 16 hours, with the plate stirring at 80 rpm and at room temperature;
Take 60 μl of the supernatant;
Add 20 μl of Laemmli 4×;
Place 50 μl on SDS PAGE gel.

The results are set out in Table 3 below:

TABLE 3
Effect of different cofactors on the activity of bromelain on wine.

| | Wine + | Wine + | Wine + | Wine + |
|---|---|---|---|---|
| | Lyophilisate | Lyophilisate + | Lyophilisate + | Lyophilisate + |
| Samples | without cofactor | 10 mM cysteine | 10 mM [symbol] | 10 mM methionine |
| Reduction of Wine | 14 | 100 | 100 | 86 |
| Proteins based on a | | | | |
| control wine | | | | |

[symbol]

indicates data missing or illegible when filed

In addition, the CaCl₂) cofactor has also been tested and shows a positive effect on the hydrolysis of wine proteins even though this result is not presented here.

Conclusion: These results validate the fact that cysteine and CaCl₂), but also and for the first time glutathione and methionine can be used as cofactors of bromelain, and by way of generalization of cysteine proteases, for protein hydrolysis. These results pave the way for new uses of cysteine proteases and their cofactors for protein hydrolysis, particularly in the context of protein stabilization in beverages based on plant musts.

1. A method of using an additive comprising using a cysteine protease and a cofactor for improving quality of beverages based on plant musts by increasing protein hydrolysis.

2. The method according to claim 1, wherein said cysteine protease is plant-derived.

3. The method according to claim 2, wherein said cysteine protease is selected from bromelain or cysteine protease extracted from ginger.

4. The method according to claim 3, wherein said bromelain is extracted from pineapple fruit.

5. The method according to claim 1, wherein said cofactor is selected from a sulphur compound, including one of cysteine, glutathione or methionine, and a calcium salt, including calcium chloride.

6. The method according to claim 1, wherein said beverage is selected from beer, wine, cider or fruit juice.

7. The method according to claim 1, wherein said additive improves protein stability and/or alcoholic fermentation of said beverage.

8. The method according to claim 1, wherein said cysteine protease and said cofactor are added in free form in said beverages.

9. The method according to claim 1, wherein said cysteine protease or said cofactor is immobilized on a support.

10. An additive for improving quality of beverages based on plant musts, the additive comprising a cysteine protease and a sulphur compound other than cysteine.

11. The additive according to claim 10, wherein said sulphur compound is selected from glutathione and methionine.

12. A method for preparing an additive for improving quality of beverages based on plant musts, the method comprising the following steps:
(a) grinding and extracting fresh pineapple fruit with a buffer at a pH above 5, and a sulphur-containing amino acid or a peptide comprising a sulphur-containing amino acid;
(b) centrifuging at a temperature ranging from 2° C. to 8° C.;
(c) clarification; and
(d) ultrafiltration.

13. A method comprising improving quality of beverages based on plant musts by increasing protein hydrolysis, through adding a dose ranging from 1 mg/L to 10,000 mg/L of an additive comprising a cysteine protease and a cofactor, for one litre of fruit juice, cider, beer or wine.

14. The method according to claim 13, wherein said additive comprises bromelain extracted from fruit and a cofactor selected from a sulphur compound, including cysteine, glutathione or methionine, and a calcium salt, including calcium chloride.
